(19) United States
(12) Patent Application Publication
(10) Pub. No.: US 2011/0207984 A1
(43) Pub. Date: Aug. 25, 2011

(54) ADDITIVE WITH MULTIPLE SYSTEM OF ZEOLITES AND METHOD OF PREPARATION

(75) Inventors: Marlon Brando Bezerra De Almeida, Rio de Janeiro (BR); Alexandre de Figueiredo Costa, Rio de Janeiro (BR); Lam Yiu Lau, Rio de Janeiro (BR); Sergio Augusto Santos Rodrigues, Rio de Janeiro (BR); Marcelo Andre Torem, Rio de Janeiro (BR)

(73) Assignee: PETROLEO BRASILEIRO S.A. - PETROBRAS, Rio de Janeiro, RJ (BR)

(21) Appl. No.: 13/059,377

(22) PCT Filed: Sep. 18, 2009
(86) PCT No.: PCT/GB2009/002254

§ 371 Date: May 13, 2011

(30) Foreign Application Priority Data
Sep. 19, 2008 (BR) PI 0803617-9

(51) Int. Cl. C07C 4/06; B01J 29/80; B01J 37/04; B01J 37/08; B01J 37/06

(52) U.S. Cl. 585/648; 502/67

(57) ABSTRACT

Additives for mixing with the catalyst inventory in process units for fluid catalytic cracking (FCC) for maximizing the production of LPG and light olefins are described. Said additives comprise a matrix that incorporates a zeolite of type MFI, preferably zeolite ZSM-5, a zeolite of type Y, and a source of phosphorus, in a single particle. Mixing of the additive in proportions from 1.0 to 40 wt. % with the equilibrium catalyst of an FCC unit maximizes the production of LPG and light olefins, chiefly propylene.

### FIELD OF THE INVENTION

The present invention relates to a catalytic system for use in units for fluid catalytic cracking of hydrocarbons, more specifically to additives comprising a matrix, a zeolite of type MFI, preferably ZSM-5, a zeolite of type Y, and a source of phosphorus, in a single particle. Said additives can be used, in combination with conventional FCC catalysts, in units for fluid catalytic cracking in such a way that the degree of conversion is maintained and there is an increase in the levels of yield of LPG, ethylene, propylene and butylenes produced.

### BACKGROUND OF THE INVENTION

The fluid catalytic cracking (FCC) process is one of the main petroleum refining technologies used throughout the world. This process makes it possible to convert a stream of hydrocarbons of high molecular weight into streams of light hydrocarbons, with greater added value, for example gasoline and liquefied petroleum gas (LPG).

In a conventional FCC process the catalyst circulates continuously in a reactor, at temperatures in the range from 480° C. to 550° C.; and in a regenerator where, in the presence of air, the coke deposited on the catalyst is burnt at temperatures in the range from 650° C. to 730° C. Traditionally, the catalyst employed in the FCC process contains a zeolite Y, alumina, kaolin and binder.

With the growth in demand for petrochemical raw materials, mainly propylene, numerous studies have been conducted with the aim of maximizing the yield of light olefins in FCC processes.

At present, an increase in the content of light olefins in the FCC process can be obtained by making changes to its operating conditions and by using different catalytic systems.

Practical experience has shown that an increase in the severity of the operating conditions in FCC processes, such as increasing the reaction temperature or increasing the catalyst/oil ratio, results in an increase in the yield of light olefins.

Although extensively investigated, maximization of light olefins by increasing the severity of the operating conditions, more specifically by increasing the temperature, leads to a great many drawbacks, such as: the need for greater circulation of catalyst, which leads to instability in flow of the catalyst and alteration of the pressure profile in the reactor; reduction in selectivity of the cracking reactions and increase in the yield of undesirable products, such as methane and ethane.

In view of the foregoing, another means employed to promote maximization of light olefins in FCC processes is modification of the catalytic systems used.

The specialist literature has various examples of modifications of zeolites selective for light olefins, such as ZSM-5, for improving the activity, selectivity and stability in processes of fluid catalytic cracking, such as the patent documents cited below.

The use of compounds containing phosphorus in the formulation of catalysts, for example, improves the performance of zeolites selective for light olefins, as can be seen in documents U.S. Pat. No. 4,605,637 and U.S. Pat. No. 4,724,06.

The use of additives based on zeolites of the mordenite type, more specifically dealuminated mordenite zeolite, incorporated in an amorphous matrix, with the aim of increasing the production of C₃ and C₄ compounds, particularly isobutane, from cracking of heavy petroleum fractions, is already proposed in document EP 0288363.

U.S. Pat. No. 6,355,591 describes the use of aluminium phosphate and zeolites of type ZSM-5, Beta, mordenite, or mixtures thereof, in the composition of additives for FCC catalysts, with the object of increasing the production of LPG.

Although the use of zeolites of type ZSM-5 in FCC processes with the objective of maximizing the production of LPG and light olefins has been studied extensively, their application as additives still comes up against limitations. The main limitation in the use of catalysts based on zeolites of type ZSM-5 as additives is that their use in large quantities leads to dilution of the base catalyst, and therefore a drop in activity of the catalytic system, also known as the dilution effect.

The activity of the catalytic system can be increased by the introduction of an active matrix such as alumina in the additive, but the alumina captures phosphorus, which is necessary for stabilization of the ZSM-5, leading to lower production of light olefins.

Another method for increasing the activity of the system could be to increase the amount of zeolite Y in the catalytic system. However, the amount of Y to be added to the base catalyst will always be limited by the physical properties of the catalyst, such as resistance to abrasion.

It must also be pointed out that an excess of zeolite Y, although increasing the activity of the catalytic system, will promote the transfer of hydrogen and lower the selectivity for the precursors of light olefins.

Document WO 2006/050487 describes the optimization of formulations of mixtures of two types of different particles, one containing zeolite of type Y, the base catalyst, and the other containing the pentasil zeolite, preferably ZSM-5, the additive. This formulation is directed at obtaining high yields of LPG and propylene. In this case, there would not be an improvement in the composition of the additive or its components.

Accordingly, to increase the yield of LPG and light olefins, it is desirable for the additive to be able to be added in larger amounts than those used at present without causing dilution of the catalytic system, interfering with its physical properties or increasing the severity of the operating variables involved.

### SUMMARY OF THE INVENTION

In the refining of petroleum, maximization of light olefins in units for the fluid catalytic cracking (FCC) process can be carried out advantageously by the addition of additives to the equilibrium catalyst inventory.

The present invention provides additives prepared from a matrix, in the form of microspheres, incorporating:
a) a zeolite of type MFI, preferably ZSM-5, at a concentration in the range from 10 to 55 wt. %;
b) a zeolite of type Y, in a proportion by weight from 0.1 to 2.0, relative to the zeolite ZSM-5;
c) phosphorus, expressed in the form of pentoxide, at concentrations between 2.0 and 25 wt. %.

Said additives can be mixed with the equilibrium catalyst inventory of an FCC unit in amounts greater than those currently used, without causing dilution of the catalytic system, or interfering with its physical properties, and at the same time maximizing the production of LPG and light olefins.

### DETAILED DESCRIPTION OF THE INVENTION

The present invention relates to additives for use in processes of fluid catalytic cracking and the method of preparation thereof.

Said additives are constituted of a matrix, prepared in the form of microspheres, incorporating:
a) a zeolite of type MFI, preferably ZSM-5, at a concentration between 10 and 55 wt. %;
b) a zeolite of type Y, in a proportion from 0.1 to 2.0 by weight, relative to the zeolite ZSM-5;
c) phosphorus, expressed in the form of P₂O₅, at concentrations from 2.0 to 25 wt. %.

In general, the method for the preparation of said additives comprises the following stages:
a) Prepare a matrix by mixing a sol of an inorganic oxide with an inert material;
b) Modify the matrix by adding a solution of a compound containing phosphorus;
c) Add a suspension of a zeolite of type MFI, preferably ZSM-5, to the modified matrix;
d) Add a zeolite of type Y, in the form of suspension, to the mixture obtained in c);
e) Hold the mixture obtained in d) at temperatures varying from 10° C. to 90° C., preferably from 20° C. to 40° C., for a period of time necessary for maturation thereof;
f) Optionally, carry out post-treatments such as washing and calcination operations.

Preferably, the sol of inorganic oxide for use in the method is a sol of silica, alumina or silica-alumina and the inert material, kaolin.

For modifying the matrix by incorporating phosphorus, it is recommended to add a solution of a compound selected from: phosphoric acid (H₃PO₄), phosphorous acid (H₃PO₃), salts of phosphoric acid, salts of phosphorous acid and mixtures thereof. Ammonium salts such as (NH₄)₂HPO₄, (NH₄)H₂PO₃, (NH₄)₂HPO₃, and mixtures thereof can also be used.

The percentage by weight of phosphorus, expressed in the form of P₂O₅, relative to the total weight of the additive must be in a range from 2.0% to 25.0% by weight, preferably between 3.0% and 20%, more preferably between 5.0% and 15%.

Among the type MFI zeolites for use in the method, ZSM-5 is preferably used.

The suspensions of zeolites of type MFI used typically have a solids content of from 20 mg/100 ml, to 30 mg/100 ml, preferably from 23 mg/100 ml to 27 mg/100 ml, such as around 25%, and particles with average diameter (d50) less than 3 μm.

The type Y zeolites that can be used in the preparation of said additives have a low sodium content, less than 1.5 wt. %, and a pore opening greater than or equal to 8 Å, for example zeolites of type USY and REY.

The suspensions of zeolites of type Y used typically have solids content of from 20 mg/100 ml to 30 mg/100 ml, preferably from 23 mg/100 ml to 27 mg/100 ml, such as around 25%, and particles with average diameter (d50) less than 3 μm. They must be added in such a way that the proportion, by weight, within the additive, between the type Y zeolite and the zeolite of type MFI, is in the range from 0.1 to 2, preferably from 0.2 to 1.5, more preferably from 0.4 to 1.33.

The type Y zeolite must be kept in contact with the mixture comprising the modified matrix and the zeolite of type MFI for a time greater than 15 minutes.

The final mixture, comprising the modified matrix, the zeolite of type MFI and the type Y zeolite, is then dried using a spray-dryer.

Optionally, post-treatments can be used, such as washing, to remove contaminants, and calcinations, with the aim of improving the mechanical properties of the additive produced, more specifically its resistance to abrasion.

Another aspect of the invention is an FCC process for maximizing the production of LPG and light olefins, which is controlled by the addition of an additive to the equilibrium catalyst inventory of the process.

The process applies to typical feeds of FCC processes, such as petroleum distillates or residual feeds, preferably feeds of the gas oil type, vacuum gas oils, atmospheric residues, and vacuum residues, typically feeds with boiling points above 343° C.

In a conventional FCC process unit, the operating conditions include: catalyst/oil ratio between 0.5:1 and 15:1, preferably between 3:1 and 8:1; time of contact with catalyst between 0.1 and 50 seconds, preferably between 0.5 and 5 seconds, and more preferably between 0.75 and 4 seconds; and reactor top temperature between 482° C. and about 565° C.

Still in relation to the FCC process unit, any commercial catalyst for FCC can be used, for example those based on zeolite type Y.

Accordingly, an additive of the present invention can be added to the equilibrium catalyst inventory of an FCC process, with the objective of maximizing the production of LPG and light olefins. This mixture must have proportions of additive in the range between 1 and 40 wt. % relative to the equilibrium catalyst inventory of the unit.

It should be pointed out that the yields of LPG and light olefins, more specifically propylene, increase significantly when we use the additive containing zeolites of type Y and of type MFI in a single particle, as demonstrated in the examples given below.

In the catalytic test in Example 2, it is observed that the use of the additive containing zeolites USY and ZSM-5, in a proportion of 10 wt. % relative to the equilibrium catalyst, leads to an absolute increase in yield of LPG and propylene, relative to the base catalyst (E-cat), of 7.1 and 4.0, respectively. In contrast, an increase in LPG and propylene, relative to the base catalyst (E-cat), is 4.8 and 2.9, respectively, when using a conventional additive containing only a zeolite of type ZSM-5 (R1), in the same proportion.

The type Y zeolite present in the additives described here is probably transformed, for the most part, to an amorphous active material, since it does not display crystallinity measurable by X-ray diffraction after hydrothermal deactivation. Accordingly, it is believed that the type Y zeolite generates precursors, which are then cracked by the type MFI zeolites, leading to an increase in the production of light olefins (C3-C4) and LPG.

It is important to emphasize that the use of high contents of a conventional additive containing only ZSM-5 generally leads to a decrease in conversion on account of the dilution effect. Now, the use of an additive containing zeolites USY and ZSM-5, as taught in the present invention, used in a proportion greater than or equal to that of a conventional additive relative to the base catalyst, leads to maintenance of conversion, without observing the dilution effect. This is clearly demonstrated in Example 6 below, where the use of 6.2% w/w of a conventional additive (R3), relative to the equilibrium catalyst, leads to a decrease in conversion. Now, the use of 10% w/w of the additive containing zeolites USY and ZSM-5 (A8) showed conversion similar to the basic case.

The examples presented below illustrate the preparation of the additive described above and its application by mixing with the equilibrium catalyst inventory in FCC processes, but these examples do not limit the scope of the invention.

### Example 1

This example illustrates the preparation of an additive containing a zeolite of type Y and a zeolite of type ZSM-5 and its physical properties.

A suspension of zeolite of type ZSM-5, where the average particle size (d50) of the zeolite was less than 3 micrometres, was prepared.

In parallel, a matrix was prepared comprising a sol of silica with alumina, to which an inert material was added, in this case kaolin.

Next, phosphorus was incorporated in the matrix formed by the addition of phosphoric acid, and then a suspension of a zeolite ZSM-5, with about 25% solids content, was added to the modified matrix.

A second zeolitic structure, of type Y, in the form of a suspension, with average particle size between 2 μm and 3 μm and solids content of 25%, was added to the mixture thus formed.

The type Y zeolite used has a low sodium content (<1.3 wt. %) and a silica-alumina framework ratio above 7, preferably around 10 or more, known by a person skilled in the art as USY.

The final mixture formed was held at temperatures varying from 20° C. to 40° C., for a period of time necessary for maturation thereof.

The mixture was then dried in a spray-dryer.

Table 1 gives the chemical compositions and properties of two additives, additive R1, containing 25 wt. % of ZSM-5, taken here as reference, and additive A1, prepared according to the present invention, containing 25 wt. % of ZSM-5 and 25 wt. % of USY.

TABLE 1

| | R1 | A1 |
|---|---|---|
| Composition, % w/w | | |
| SiO₂ | 55.4 | 63.4 |
| Al₂O₃ | 26.2 | 19.0 |
| Na₂O | 0.11 | 0.30 |
| P₂O₅ | 15.5 | 15.6 |
| Properties before deactivation | | |
| Apparent density g/ml | 0.71 | 0.71 |
| Specific area, m²/g | 72 | 92 |
| Area of zeolite, m²/g | 58 | 78 |
| Mesoporous area, m²/g | 14 | 14 |
| Properties after deactivation (815° C./5 h) | | |
| Specific area, m²/g | 99 | 133 |
| Area of zeolite, m²/g | 60 | 68 |
| Mesoporous area, m²/g | 39 | 65 |

These characteristics show that additive A1, prepared according to the present invention, has a density similar to the reference additive, but has a greater specific area, both before and after the hydrothermal deactivation.

### Example 2

This example compared the conversion and the yields of the products obtained in a catalytic test for a reference additive (R1) and an additive (A1), both described in Example 1.

The additives to be tested were treated beforehand with 100% steam at 815° C. for 5 h.

Each additive treated was then mixed with an equilibrium catalyst (E-cat), obtained from a commercial FCC unit, in a proportion by weight of 10% of additive to 90% of E-cat.

Table 2 shows the chemical composition and physical properties of the equilibrium catalyst.

TABLE 2

| | E-cat |
|---|---|
| Composition | |
| SiO₂, % w/w | 55.3 |
| Al₂O₃, % w/w | 40.1 |
| Na₂O, % w/w | 0.53 |
| RE₂O₃% w/w | 2.48 |
| P₂O₅% w/w | 0.60 |
| Ni, mg/kg | 3579 |
| V, mg/kg | 3074 |
| Physical Properties | |
| Specific area, m²/g | 142 |
| Area of zeolite, m²/g | 112 |
| Mesoporous area, m²/g | 30 |

The mixtures comprising the respective additives and the equilibrium catalyst, in the proportions described above, were tested in an ACE laboratory unit (Kaiser Technology, U.S. Pat. No. 6,069,012) using heavy gas oil as feed (properties presented in Table 3), catalyst/oil weight ratio 6 and temperature 535° C.

TABLE 3

| | |
|---|---|
| ° API | 19.2 |
| Density | 0.935 |
| Aniline Point (° C.) | 83.3 |
| Total Sulphur (wt. %) | 0.57 |
| Total Nitrogen (mg/kg) | 2835 |
| Basic Nitrogen (mg/kg) | 854 |
| RCR (%) | 0.55 |

Table 4 shows the comparative results for conversion and yield for the equilibrium catalyst, and for mixtures of the equilibrium catalyst with the additives described in Example 1 (R1 and A1).

The values of conversion and yield for the mixtures presented in Table 4 are the absolute differences between the base values, conversion and yield when using the equilibrium catalyst without additives, and the values obtained with the mixtures (E-cat+additives).

TABLE 4

| | E−cat + R1 ⁽²⁾ | E−cat + A1 ⁽²⁾ |
|---|---|---|
| Conversion % w/w ⁽¹⁾ | −1.1 | −0.8 |
| Yields % w/w ⁽¹⁾ | | |
| Fuel gas | 0.2 | 0.2 |
| LPG | 4.8 | 7.1 |
| Propane | 0.4 | 0.4 |
| Propylene | 2.9 | 4.0 |
| n-Butane | 0.0 | 0.0 |
| Isobutane | 0.6 | 0.7 |
| C₄ Olefins | 1.0 | 2.0 |
| Gasoline | −5.8 | −7.7 |
| LCO | −0.7 | −0.7 |
| Decanted oil | 1.9 | 1.5 |
| Coke | −0.4 | −0.5 |

⁽¹⁾ Absolute differences relative to the value obtained with the pure equilibrium catalyst (100%), taken here as reference.
⁽²⁾ Mixture containing 90 wt. % of equilibrium catalyst and 10 wt. % of additive.

These results show that additive A1, containing 25 wt. % of USY and 25 wt. % of ZSM-5, prepared according to the method described in Example 1, gives a higher yield of propylene and LPG than the additive containing only zeolite ZSM-5 (R1).

### Example 3

This example illustrates the conversion and the yields of the products obtained in a catalytic test for a reference additive (R1), described in Example 1, and for another three additives (A2, A3 and A4), prepared according to the method described in Example 1.

Additives A2-A4 contain 25 wt. % of ZSM-5 and 25 wt. % of USY, with only the composition of the matrix varying. The reference additive R1 contains only zeolite ZSM-5 at a concentration of 25 wt. %.

The additives to be tested undergo pretreatment, deactivation, with 100% steam at 815° C. for 5 h.

Table 5 shows the properties and chemical composition of the additives.

Each additive treated was then mixed with an equilibrium catalyst (E-cat), obtained from a commercial FCC unit, in a proportion by weight of 10% of additive to 90% of E-cat.

The mixtures, comprising the respective additives and the equilibrium catalyst, were tested in an ACE laboratory unit (Kaiser Technology, U.S. Pat. No. 6,069,012) using heavy gas oil as feed (properties presented in Table 3), catalyst/oil weight ratio 6 and temperature 535° C.

TABLE 5

| | R1 | A2 | A3 | A4 |
|---|---|---|---|---|
| Composition % w/w | | | | |
| SiO₂ | 55.4 | 60.6 | 62.3 | 62.9 |
| Al₂O₃ | 26.2 | 22.7 | 22.5 | 23.6 |
| Na₂O | 0.11 | 0.66 | 0.38 | 0.35 |
| P₂O₅ | 15.5 | 13.1 | 13.3 | 11.3 |
| Properties before deactivation | | | | |
| Specific area, m²/g | 72 | 87 | 94 | 104 |
| Area of zeolite, m²/g | 58 | 65 | 70 | 80 |
| Mesoporous area, m²/g | 14 | 22 | 24 | 24 |
| Properties after deactivation | | | | |
| Specific area, m²/g | 99 | 93 | 108 | 133 |
| Area of zeolite, m²/g | 60 | 58 | 64 | 76 |
| Mesoporous area, m²/g | 39 | 35 | 44 | 58 |

Table 6 gives the yields and the conversion achieved with the reference additive (R1) and additives A2-A4, when used in an FCC process, in the conditions described above.

TABLE 6

| | E-cat + | E-cat + | E-cat + | E-cat + |
|---|---|---|---|---|
| | R1 | A2 | A3 | A4 |
| Conversion, % w/w | 63.5 | 64.1 | 64.0 | 64.7 |
| Yields, % w/w | | | | |
| Fuel gas | 3.60 | 3.93 | 3.90 | 3.57 |
| Hydrogen | 0.20 | 0.19 | 0.19 | 0.18 |
| Methane | 1.13 | 1.14 | 1.15 | 1.12 |
| Ethane | 0.76 | 0.74 | 0.74 | 0.71 |
| Ethylene | 1.52 | 1.86 | 1.83 | 1.56 |
| LPG | 22.0 | 23.2 | 23.1 | 22.7 |
| Propane | 1.89 | 2.06 | 2.04 | 1.94 |
| Propylene | 8.75 | 9.23 | 9.25 | 8.92 |
| n-Butane | 0.82 | 0.88 | 0.87 | 0.87 |
| Isobutane | 4.24 | 4.72 | 4.67 | 4.69 |
| C₄ Olefins | 6.29 | 6.32 | 6.28 | 6.34 |
| Gasoline | 32.9 | 31.6 | 31.8 | 33.1 |
| LCO | 19.8 | 18.7 | 18.9 | 18.9 |
| Decanted oil | 16.7 | 17.2 | 17.0 | 16.4 |
| Coke | 4.9 | 5.4 | 5.2 | 5.3 |
| C₃⁼/Conversion, % w/w | 13.8 | 14.4 | 14.4 | 13.8 |
| LPG/Conversion, % w/w | 34.7 | 36.2 | 36.1 | 35.2 |

These results demonstrate that additives A2-A4 give higher conversion, when using the same catalyst/oil ratio, as R1 and that they have a selectivity for LPG better than that of R1.

Additives A2 and A3 stand out by the preferential improvement in selectivity for light olefins, and additive A4 by the improvement in conversion.

### Example 4

This example illustrates the conversion and the yields of products obtained in a catalytic test for a reference additive (R2) and an additive (A5), the latter prepared according to the method described in Example 1.

Additive A5 contains 35 wt. % of ZSM-5 and 15 wt. % of USY. The reference additive R2 contains only zeolite ZSM-5 at a concentration of 35 wt. %.

The additives to be tested underwent pretreatment, deactivation, with 100% steam at 815° C. for 5 h.

The properties of the additives are shown in Table 7.

TABLE 7

| | R2 | A5 |
|---|---|---|
| Composition, % w/w | | |
| SiO₂ | 61.4 | 65.5 |
| Al₂O₃ | 23.2 | 21.5 |
| Na₂O | 0.08 | 0.18 |
| P₂O₅ | 13.2 | 11.1 |
| Properties before deactivation | | |
| Specific area, m²/g | 116 | 131 |
| Area of zeolite, m²/g | 87 | 97 |
| Mesoporous area, m²/g | 29 | 34 |
| Properties after deactivation | | |
| Specific area, m²/g | 124 | 140 |
| Area of zeolite, m²/g | 75 | 76 |
| Mesoporous area, m²/g | 49 | 64 |

Each treated additive was then mixed with an equilibrium catalyst (E-cat), obtained from a commercial FCC unit, at a weight ratio of 10% of additive to 90% of E-cat.

The mixtures comprising the respective additives and the equilibrium catalyst, in the proportions described above, were tested in an ACE laboratory unit (Kaiser Technology, U.S. Pat. No. 6,069,012) using heavy gas oil as feed (properties presented in Table 3), catalyst/oil weight ratio 6 and temperature 535° C.

Table 8 gives the results of conversion and of yield obtained for additives R2 and A5 in an FCC process.

These results demonstrate that additive A5 gives higher conversion and higher selectivity for LPG, when using the same catalyst/oil ratio, as the reference additive R2

TABLE 8

| | E-cat + R2 | E-cat + A5 |
|---|---|---|
| Conversion % w/w | 62.8 | 64.4 |
| Yields % w/w | 4.60 | 4.13 |
| Fuel gas | 4.60 | 4.13 |
| Hydrogen | 0.24 | 0.18 |
| Methane | 1.07 | 1.13 |
| Ethane | 0.83 | 0.74 |
| Ethylene | 2.46 | 2.07 |
| LPG | 22.1 | 23.8 |
| Propane | 2.23 | 2.19 |
| Propylene | 9.16 | 9.46 |
| n-Butane | 0.85 | 0.89 |
| Isobutane | 3.80 | 4.92 |
| C₄ Olefins | 6.04 | 6.31 |
| Gasoline | 30.4 | 31.3 |
| LCO | 19.7 | 18.8 |
| Decanted oil | 17.5 | 16.8 |
| Coke | 5.7 | 5.2 |
| C₃⁼/Conversion % w/w | 14.6 | 14.7 |
| LPG/Conversion % w/w | 35.2 | 36.9 |

### Example 5

This example illustrates the use of zeolites USY and REY as source of zeolite type Y in the preparation of additives according to the present invention, as well as their characterization and use in FCC processes.

Additives A6 and A7 are prepared by the method described in Example 1, additive A6 having concentrations by weight of 20% of USY and 25% of ZSM-5 and additive A7 having concentrations by weight of 20% of REY and 25% of ZSM-5.

Zeolite REY was obtained by ion exchange of zeolite Y with ammonia and solution of rare earths so as to obtain 2% RE₂O₃ in the zeolite. Then the zeolite underwent calcination at a temperature close to 500° C. and was then incorporated in an additive using the procedure described in Example 1.

The additives to be tested underwent pretreatment, deactivation, with 100% steam at 815° C. for 5 h.

Table 9 shows the composition and properties of the additive.

TABLE 9

| | A6 | A7 |
|---|---|---|
| Composition % w/w | | |
| SiO₂ | 58.8 | 58.2 |
| Al₂O₃ | 22.4 | 22.3 |
| Na₂O | 0.38 | 0.46 |
| P₂O₅ | 16.6 | 17.0 |
| RE₂O₃ | 0.00 | 0.21 |
| Properties before deactivation | | |
| Surface area, m²/g | 82 | 76 |
| Area of zeolite, m²/g | 65 | 62 |
| Mesoporous area, m²/g | 17 | 15 |
| Properties after deactivation | | |
| Surface area, m²/g | 105 | 103 |
| Area of zeolite, m²/g | 62 | 63 |
| Mesoporous area, m²/g | 43 | 40 |

Each treated additive was then mixed with an equilibrium catalyst (E-cat), obtained from a commercial FCC unit, at a weight ratio of 10% of additive to 90% of E-cat.

The mixtures comprising the respective additives and the equilibrium catalyst, in the proportions described above, were tested in an ACE laboratory unit (Kaiser Technology, U.S. Pat. No. 6,069,012) using heavy petroleum as feed (properties presented in Table 3), catalyst/oil weight ratio 6 and temperature 535° C.

Table 10 shows the results for yield and conversion obtained with additives A6 and A7 in an FCC process.

TABLE 10

| | E-cat + A6 | E-cat + A7 |
|---|---|---|
| Conversion, % w/w | 64.0 | 63.9 |
| Yields, % w/w | | |
| Fuel gas | 2.82 | 2.90 |
| Hydrogen | 0.21 | 0.27 |
| Methane | 1.03 | 0.98 |
| Ethane | 0.62 | 0.66 |
| Ethylene | 0.96 | 0.98 |
| LPG | 17.2 | 17.1 |
| Propane | 1.42 | 1.32 |
| Propylene | 6.02 | 6.35 |
| n-Butane | 0.74 | 0.70 |
| Isobutane | 3.59 | 3.30 |
| C₄ Olefins | 5.42 | 5.41 |
| Gasoline | 37.3 | 37.3 |
| LCO | 18.7 | 19.1 |
| Decanted oil | 17.3 | 17.0 |
| Coke | 6.7 | 6.7 |
| C₃⁼/Conversion, % w/w | 9.4 | 9.9 |
| LPG/Conversion, % w/w | 26.8 | 26.7 |

These results demonstrate that additive A6 gives performance similar to A7. This implies that zeolite USY can be used in the preparation of additives according to the present invention, without resulting in losses of yield and conversion when compared with the use of zeolite REY.

### Example 6

This example illustrates the conversion and the yields of the products obtained in a catalytic test for a reference additive (R3) and an additive (A8), prepared according to the method described in Example 1, in order to demonstrate the dilution effect.

The commercial additive R3, with high content of ZSM-5, was prepared by the conventional method without the addition of zeolite USY.

The additives to be tested were treated beforehand with 100% steam at 815° C. for 5 h.

Each treated additive was then mixed with an equilibrium catalyst (E-cat), obtained from a commercial FCC unit, in a proportion by weight of 6.2% of additive R3 to 93.8% of E-cat and 10% of additive A8 to 90% of E-cat, resulting in the same content of ZSM-5 in the mixture.

The mixtures comprising the respective additives and the equilibrium catalyst, in the proportions described above, were tested in an ACE laboratory unit (Kaiser Technology, U.S. Pat. No. 6,069,012) using heavy gas oil as feed (properties presented in Table 3), catalyst/oil weight ratio 5 and temperature 535° C.

The results of the catalytic tests are shown in Table 11.

Additive R3, with high content of ZSM-5, prepared by the conventional method without USY applied at lower contents in the mixture leads to a decrease in total conversion (dilution effect). This was demonstrated when 6.24% of additive R3 was added to the system and the conversion fell from 60.6% to 59.2%.

In the case of the novel additives proposed, the application of 10% of A8 results in a conversion equivalent to the use of the equilibrium catalyst without additive (E-cat). The selectivity for propylene and LPG of A8 is higher than the selectivity of R3, with both systems having the same content of ZSM-5.

TABLE 11

| | E-cat | E-cat + R3 | E-cat + A8 |
|---|---|---|---|
| Conversion, % w/w | 60.6 | 59.2 | 60.5 |
| Yields, % w/w | | | |
| Fuel gas | 2.48 | 2.73 | 3.50 |
| Hydrogen | 0.19 | 0.24 | 0.22 |
| Methane | 0.99 | 0.91 | 0.94 |
| Ethane | 0.73 | 0.56 | 0.61 |
| Ethylene | 0.57 | 1.03 | 1.73 |
| LPG | 10.4 | 16.7 | 20.1 |
| Propane | 0.92 | 1.28 | 1.69 |
| Propylene | 3.08 | 6.53 | 8.20 |
| n-Butane | 0.61 | 0.63 | 0.70 |
| Isobutane | 2.28 | 3.00 | 3.55 |
| C₄ Olefins | 3.56 | 5.26 | 5.96 |
| Gasoline | 41.2 | 33.9 | 31.0 |
| LCO | 20.9 | 20.6 | 19.8 |
| Decanted oil | 18.5 | 20.2 | 19.7 |
| Coke | 6.5 | 5.9 | 5.9 |
| C₃⁼/Conversion | 5.1 | 11.0 | 13.5 |
| LPG/Conversion | 17.2 | 28.2 | 33.2 |

### SUMMARY

The present invention relates to an additive with multiple system of zeolites for fluid catalytic cracking units, characterized in that it comprises a matrix, in the form of microspheres, incorporating:
a) a zeolite of type MFI, at a concentration in the range from 10 to 55 wt. %;
b) a zeolite of type Y, in proportion by weight between 0.1 and 2.0 relative to the zeolite of type MFI;
c) the chemical element phosphorus, at a concentration between 2.0 and 25 wt. % of pentoxide.

1. Additive for use in fluid catalytic cracking units, characterized in that it comprises a matrix, in the form of microspheres, wherein said matrix incorporates:
a) a zeolite of type MFI, at a concentration in the range from 10 to 55 wt. % based on the matrix;
b) a zeolite of type Y, wherein the weight ratio of the type Y zeolite to the type MFI zeolite is between 0.1:1 and 2.0:1;
c) the chemical element phosphorus, wherein the amount of phosphorus corresponds to the amount present in form 2.0 to 25 wt. % of phosphorus pentoxide, based on the matrix.

2. Additive according to claim 1, characterized in that the zeolite of type MFI is zeolite ZSM-5.

3. Additive according to claim 1, characterized in that the matrix comprises a sol of inorganic oxide and an inert material.

4. Additive according to claim 3, characterized in that the sol of inorganic oxide is selected from one or more of: silica, alumina and silica-alumina.

5. Additive according to claim 3, characterized in that the inert material is kaolin.

6. Additive according to claim 1, characterized in that the weight ratio of the type Y zeolite to the zeolite of type MFI is in the range from 0.2:1 to 1.5:1.

7. Additive according to claim 6, characterized in that the weight ratio of the type Y zeolite to the zeolite of type MFI is in the range from 0.4:1 to 1.33:1.

8. Additive according to claim 1, characterized in that the amount of phosphorus corresponds to the amount present in from 3.0 to 20 wt. % of phosphorus pentoxide, based on the matrix.

9. Additive according to claim 8, characterized in that the amount of phosphorus corresponds to the amount present in from 5.0 to 15.0 wt. % of phosphorus pentoxide, based on the matrix.

10. Additive according to claim 1, characterized in that the type Y zeolite comprises the chemical element sodium at a concentration below 1.5 wt. % and has a pore diameter of greater than or equal to 8 Å.

11. Additive according to claim 1, characterized in that the chemical element phosphorus is incorporated by adding to the matrix a compound selected from: phosphoric acid (H₃PO₄), phosphorous acid (H₃PO₃), salts of phosphoric acid, salts of phosphorous acid or ammonium salts of the type (NH₄)₂HPO₄, (NH₄)H₂PO₃, (NH₄)₂HPO₃, or mixtures thereof in any proportions.

12. Additive according to claim 1, characterized in that the type Y zeolite used has an average particle size of from 2 μm to 3 μm.

13. Additive according to claim 1, characterized in that the average particle diameter of the type MFI zeolite is less than 3 μm.

14. Method for preparation of an additive as defined in claim 1, characterized in that it comprises the following stages:
a) Preparing a matrix by mixing a sol of an inorganic oxide with an inert material;
b) Modifying the matrix by adding a solution of a compound containing the chemical element phosphorus;
c) Adding a suspension of a zeolite of type MFI, having a solids content of around 25 mg/100 ml, to the modified matrix, thus obtaining a mixture;
d) Adding a zeolite of type Y, in the form of powder or suspension having a solids content of 10 around 25 mg/100 ml, to the mixture obtained in step (c);
e) Holding the mixture obtained in (d) at one or more temperatures in the range from 10° C. to 90° C., for a period of time necessary for maturation thereof;
f) Optionally, carrying out one or more post treatments such as washing and calcination operations.

15. A method according to claim 14, wherein the suspension of a zeolite of type MFI and the suspension of a zeolite of type Y each independently have a solids content of from 20 mg/100 ml to 30 mg/100 ml, preferably from 23 mg/100 ml to 27 mg/100 ml.

16. A method according to claim 14 or claim 15, characterized in that the temperature range necessary for maturation of the mixture obtained in (d) is from 20° C. to 40° C.

17. A method according to any one of claims 14 to 16 claim 14, characterized in that the duration of stage (d) is greater than 15 minutes.

18. FCC process for maximization of LPG and olefins in conditions of cracking, characterized in that an additive as defined in claim 1 is mixed, in proportions between 1.0 and 40 wt. %, with the equilibrium catalyst inventory of the process.

19. A process according to claim 18, which further comprises isolating one or more components of the FCC output.

20. FCC process for maximization of LPG and olefins in conditions of cracking, characterized in that an additive obtained by a method according to claim 14 is mixed, in proportions between 1.0 and 40 wt. %, with the equilibrium catalyst inventory of the process.
